(19) United States
(12) Patent Application Publication
(10) Pub. No.: US 2012/0207688 A1
(43) Pub. Date: Aug. 16, 2012

(54) Rosacea Topical Skin Treatment Method and Formulation

(76) Inventor: B. Eugene Guthery, Texarkana, TX (US)

(21) Appl. No.: 13/503,783

(22) PCT Filed: Oct. 27, 2010
(86) PCT No.: PCT/US10/54322

§ 371 Date: Apr. 24, 2012

(30) Foreign Application Priority Data
Oct. 28, 2009 (US) 61255556

(51) Int. Cl. A61K 31/555; A61K 33/30; A61K 38/57; A61K 38/07; A61K 31/355; A61K 31/375; A61K 8/49; A61K 8/58; A61Q 17/04; A61K 8/92; A61P 17/00; A61P 17/10; A61P 29/00; A61P 39/06; A61K 31/351

(52) U.S. Cl. 424/59; 514/188; 514/460; 424/642; 514/18.7; 514/458

(57) ABSTRACT

A method for topical treatment of rosacea comprising application of a formulation to the affected area is disclosed. The formulation comprises one or more iron chelators. Omadine, a bispyrithione salt, and kojic acid are the preferred iron chelators. The formulation also comprises one or more false substrates for arachidonic acid. The preferred false substrates for arachidonic acid are alpha-linoleic acid and gamma dihomo-linolenic acid. The formulation further comprises an inhibitor of stratum corneum tryptic enzyme (SCTE). A preferred stratum corneum tryptic enzyme inhibitor is zinc. The formulation also preferably includes one or more medium-chain saturated fatty acid monoester. The preferred medium-chain fatty acid monoesters are glycerol monolaurate and glycerol monocaprylate. Formulation components are solubilized in a suitable carrier base which includes emollient, humectant, antioxidant and sunscreen components. The method preferably comprises application of a leave-on formulation. Alternatively, a pre-step of application of a rinse-off formulation containing a higher concentration of an iron chelator may be employed in the regimen.

### CROSS REFERENCE TO RELATED APPLICATIONS

This application claims the benefit of U.S. Provisional Application No. 61/255,556 filed on 28 Oct. 2009.

### TECHNICAL FIELD OF INVENTION

This invention relates generally to methods and compositions for topical application to the skin to treat rosacea, also known as acne rosacea.

### BACKGROUND OF THE INVENTION

Rosacea, or acne rosacea, is a chronic and recurrent inflammatory skin disease affecting the central face and/or V-area of the chest. It is characterized by frequent flushing, erythema, and telangiectasia (small dilated blood vessels near the surface of the skin) interspersed with episodes of inflammation during which swelling, papules and pustules, and occasionally, nodules, are evident.

Rosacea affects approximately 14 million American people. It is often exhibited by people of northern European heritage, and is rarely exhibited by dark-skinned individuals. It is most prevalent between the third and fourth decades of life, peaking between the ages of forty and fifty years. It affects more women than men at a ratio of three to one.

Patients with rosacea experience periods of remission and relapse. In some, the disease progresses sequentially through stages. Episodic erythema may precede the first stage. This includes blushing and flushing that reportedly may be provoked by nonspecific triggers. Eventually, a dark-red erythema is chronically evident in the affected area.

Stage 1 has generally been described as persistent erythema and telangiectasia in the area of the nose, cheeks, and glabella.

Stage 2 involves a progression of the disease characterized by persistent small, firm inflammatory papules and pustules which may persist for weeks. Papules may have central necrosis and facial pores may become large and prominent, signifying fibroplasia.

Stage 3 usually is reached by a small subset of rosacea sufferers. This stage is characterized by persistent deep erythema, telangiectasia, papules, pustules, large inflammatory nodules or granulomas, and tissue hyperplasia. It may even involve development of coarse and irregular facial contours, thickened edematous skin, hypertrophy of connective tissue and sebaceous glands, and perhaps disfiguring rhinophyma.

Previously reported approaches for the treatment of rosacea have included application to the affected area of agents for cleansing of the skin or agents for treatment of other skin disorders such as acne vulgaris, some of which are non-prescription or over the counter treatment agents. However, rosacea patients often have skin which is unusually vulnerable to chemical or physical insults. Soaps, alcoholic cleansers, tinctures, astringents, abrasives, and peeling agents may aggravate rather than alleviate rosacea.

Other approaches for the treatment of rosacea include topical application of creams or ointments designated as prescription only. Some patients may be hypersensitive or allergic to such treatments, while others are tolerant.

For example, one approach for the treatment of rosacea is the application of topical antibiotics. Topical erythromycin, clindamycin, and tetracycline, usually in concentrations of 0.5% to 2.0% have been used. Imidazoles, such as Ketoconazole cream (Nizoral cream), and Metronidazole (a synthetic, nitroimidazole-derivative antibacterial and anti-protozoal agent) have been used successfully.

Another class of prescription topical agents reported for the treatment of rosacea is the retinoids. The acneiform component of rosacea may respond to tretinoin (Retin A) or retinaldehyde (0.5 to 0.10%). Isotretinoin (Accutane) has also been used for some patients. Not all patients can use retinoids as some exhibit sensitivity.

Azelaic acid has been reported as a rosacea treatment agent in a concentration of 20% in a cream base. An undesirable side-effect which has occasionally been reported with use of azelaic acid is the growth of hair on treated areas.

There remains a need for other effective topical treatments for rosacea.

### DETAILED DESCRIPTION

A method for treatment of rosacea utilizing a formulation for topical application to the skin is herein disclosed. In a first embodiment, a method and formulation comprises applying an iron-chelating formulation topically to the area afflicted with rosacea. It has now been found that chelating iron in this way is effective in reducing or eliminating the symptoms of rosacea.

A preferred regimen is also disclosed for use of the formulation. In this regimen, the formulation (a “leave-on” formulation) is applied topically to the area afflicted with rosacea and left on the area without rinsing. Repeated daily application of the leave-on formulation to the afflicted area, until the area is cosmetically acceptable to the user, may be employed. In general, the time required for cosmetically acceptable results is about three to four weeks. If desired, the formulation can be applied thereafter on a continuous daily or less frequent basis to prevent reoccurrence of iron-mediated rosacea outbreaks.

In another regimen, a step precedent to said application of the leave-on topical formulation is employed. In the step precedent, a higher concentration of iron chelator is provided in a wash-off formulation which the user may apply to, and leave in contact with, the affected area for about 20 seconds to one minute, then remove by gentle but thorough rinsing. A greater efficacy may be seen when the formulation is left in contact with the affected area for five to fifteen minutes prior to rinsing. The formulation is kept in contact with the affected skin in order to allow the formulation components to bind the iron in the area of affliction. The relative concentrations are further discussed below.

Preferred iron chelators are alkaline earth metal salts of bispyrithione. Useful bispyrithione salts (pyridinethione) are derivatives of 1-hydroxy-2-pyridinethione or the tautomeric form thereof. Various pyrithione metal salts and derivatives are known and described in numerous references such as in U. S. Pat. Nos. 2,809,971, 2,742,476, and 3,236,733. Heavy metal salts and dimeric forms of pyrithione are useful for the preparation of the formulation used in this invention. Zinc and magnesium salts of 2, 2′-dithiobis (pyridine-1-oxide) are most generally used and the zinc salts are preferred. The zinc (Zinc Omadine) and magnesium salts of bispyrithione (Omadine MDS) are both available from Arch Biocides. Omadine (bispyrithione salts—also called pyridinethione salts) has two pyrithione moieties linked via a disulfide bridge. Zinc Omadine is an effective iron chelator.

Concentrations of Zinc Omadine or Omadine MDS (Collectively “Omadine”) useful in the present invention are from 0.0001% to 5%. The most preferable concentrations of omadine are from 0.0001% to 2.0%. A 48% aqueous cosmetic grade suspension of Zinc Omadine is preferred. The concentration of Zinc Omadine in the rinse-off formulation is preferably from 1%-5%, while the concentration in the leave-on formulation is preferably from 0.0001% to 0.5%.

Omadine also may function as a preservative in the formulation due to its bactericidal activity. Thus, other commonly used preservatives which have been reported to cause contact hypersensitivity in some individuals need not be employed in the formulation.

In preparation of the formulation, it has been found that Zinc Omadine can combine with trace amounts of iron to produce a brown or yellow color which could be aesthetically unacceptable for a cosmetic composition. Zinc salts, like zinc sulfate or zinc acetate, can be advantageously added to the composition of the present invention to prevent the discoloration. Preferably, zinc salts are employed in the formulation in concentrations of 0.05 to 1.0%.

Other iron chelators which may be employed in the formulation either alone, in combination with Zinc Omadine, or in combination with each other are, kojic acid, kojic acid dipalmitate, 1,10-phenanthroline, ethylenediaminetetraacetic acid (EDTA), 2-furildioxime, desferrioxamine, desferrithiocin, desferri-exochelin, deferiprone and hydroxypyridione analogs, tackpyridine, pyridoxyl isonicitinoyl hydrazone, triapine, and 2-hydroxy-1-napthaldehyde-3-thiosemicarbazone.

In a preferred embodiment, the formulation comprises Zinc Omadine in combination with kojic acid, kojic acid dipalmitate, or another kojic acid derivative, with kojic acid dipalmitate being preferred. Kojic acid dipalmitate is an iron chelator which is also helpful in reducing redness in the skin and suppressing cellular damage. It also increases the SPF (Sun Protection Factor) value of the formulation. Preferably, kojic acid dipalmitate is employed in a concentration of from 0.5% to 6.0%. The most preferred concentration is from 1.0% to 5.0%.

Without wishing to be bound by any theory, it is believed that the skin has a significant level of iron which is normally available to participate in a reaction known as the Fenton reaction. The skin cells of patients with rosacea have been found to have higher levels of iron (stored as ferritin) than individuals without rosacea. Reactive oxygen species (ROS) or free radicals are produced via the iron dependent Fenton reaction. When the iron is chelated, the Fenton reaction cannot occur, thus stopping ROS formation. This stoppage prevents skin damage as well as rosacea.

The formulation preferably includes a false substrate for arachidonic acid metabolism. Arachidonic acid metabolism can result in inflammation. The false substrate included in the formulation of the invention is selected from eighteen to twenty carbon unsaturated long-chain fatty acids. Most preferably, the false substrate is selected from a group consisting of elaidic acid, oleic acid, gamma-dihomo-linolenic acid and linoleic acid.

Most preferably, alpha-linoleic acid is employed. Gamma-dihomo-linolenic acid, one of the omega 6 fatty acids, is useful because it is metabolized by the skin to anti-inflammatory prostaglandin E-1 (PGE1).

The preferred concentration of the unsaturated fatty acids, i.e., linoleic acid and gamma-dihomo-linolenic acid, is from 0.01% to 5.0% by weight. The most preferred concentration of these unsaturated fatty acids is from 0.10% to 5.0%.

Purified single component free fatty acids may be used but are very expensive to obtain. However, unsaturated fatty acid mixtures are readily available as hydrolysates of various oils such as linseed oil, soybean oil, borage oil and the like. These oils are often a very good source of linoleic and gamma-dihomo linolenic acids. The long-chain unsaturated fatty acids, such as linoleic acid (C18:2), provided in commercial products such as Emersol 305 or Emersol 315 (Cognis Corporation, Cincinnati, Ohio, a division of Cognis Corporation in Toronto, Canada) are preferred. Also, oleic acid, known as Emersol 221, can be substituted or added to the Emersol 315. The preferred hydrosylates should not contain linolenic acid as this compound promotes inflammation.

The formulation also preferably includes one or more medium-chain saturated fatty acid monoester. The preferred medium-chain fatty acid monoesters are glycerol monolaurate and glycerol monocaprylate, with glycerol monolaurate being the most preferred. Glycerol monolaurate provides an emollient and moisturizing effect on the skin and may advantageously provide antibacterial activity against gram-positive bacteria. The preferred range of the medium-chain fatty acid ester is from about 0.01% to about 5% by weight. The most preferred concentration is from 0.01% to about 2% by weight.

When unsaturated fatty acids are employed, an antioxidant component is added to the formulation to prevent saturation of the unsaturated fatty acids such as linoleic acid and gamma-dihomo-linolenic acid. More than one antioxidant may be employed It is most preferred to use one antioxidant that functions in the lipid phase of the formulation and another antioxidant that functions in the aqueous phase of the formulation. Suitable antioxidant choices include butylatedhydroxy toluene (BHT), Vitamin E (alpha tocopherol), ascorbic acid, and propyl gallate or mixtures containing propyl gallate, such as Tenox S-1 (ABCO Chemical Co., Kingsport, Tenn.).

A preferred antioxidant in the aqueous phase is propyl gallate. Propyl gallate may be increased above antioxidant levels to achieve a potent anti-inflammatory effect. Propyl gallate is a preferred antioxidant because, not only is it an excellent antioxidant, it also contributes to the anti-inflammatory properties by blocking lipooxygenase 5 and enhancing the sunscreen effectiveness by raising the Sun Protection Factor (SPF). Utilization of sunscreen agents may be advantageous in the treatment of rosacea, and as propyl gallate has UV blocking properties, it also functions in the formulation to prevent damage to the skin from ultraviolet light. The preferred concentration of propyl gallate is from 0.01% to 5%. The most preferred concentration of propyl gallate is from 0.10% to 1.5%.

The preferred antioxidants in the lipid phase are butylatedhydroxy toluene (BHT) and Vitamin E. The preferred concentration of BHT is from 0.01% to 5%. The most preferred concentration of BHT is from 0.10% to 1.5%. Vitamin E is used in concentrations up to 5.0%.

It is desirable in an embodiment of the method for treating rosacea to include a sunscreen agent in the rosacea treatment formulation if the formulation is employed by the user during daylight hours. This will help prevent reactions triggered by exposure to sun. Any of the additional sunscreen agents listed in the Food and Drug Administration monograph (Code of Federal Regulations-Title 21-Part 352-Sunscreen Drug Products for Over-the-Counter Human Use) revised 1 Apr. 2001 can be advantageously added to the composition of the invention. For example, the following agents can be added to the formulation: Aminobenzoic acid (PABA) up to 15%, Avobenzone up to 3%, Cinoxate up to 3%, Dioxybenzone up to 3%, Homosalate up to 15%, Menthyl anthranilate up to 5%, Octocrylene up to 10%, Octyl methoxycinnamate up to 7.5%, Octyl salicylate up to 5%, Oxybenzone up to 6%, Padimate O up to 8%, Phenylbenzimidazole sulfonic acid up to 4%, Sulisobenzone up to 10%, Titanium dioxide up to 25%, Trolamine salicylate up to 12%, and Zinc Oxide up to 25%.

Suitable sunscreens protect the skin from damage caused by ultraviolet light. Ultraviolet light is of three varieties and all are damaging to the skin. Ultraviolet B has wavelengths from 290 to 320 nm. Ultraviolet A-I has wavelengths from 340 to 400 nm. Ultraviolet A-II has wavelengths from 320 to 340 nm. It is desirable to use sunscreen combinations that cover wavelengths of light from 290 to 400 nm.

Preferably, zinc oxide will be employed in the formulation in combination with avobenzone. This combination will protect against UVB, UVA-II and UVA-I.

It is most preferred to use zinc oxide in submicroscopic size (<200 nm) so that visible light scattering is minimized and the particles appear invisible on the skin. Z-Cote and Z-Cote Max are preferred; these products are available from BASF (Shreveport, La.). At this small particle size, the particles attenuate UV light, predominantly by absorption similar to an organic sunscreen. Because the submicroscopic zinc oxide is particulate, the size precludes entry into skin.

It is preferable to choose the combination of sunscreen agents which has the lowest irritation potential when applied to irritated skin over a period of three to four weeks. Using cyclomethicone and dimethicone can advantageously abrogate the potential irritation of sunscreen agents.

Topical retinoids can decrease vascular endothelial growth factor expression by keratinocytes and may prevent skin neoangiogenesis in certain skin diseases. Retinaldehyde, a retinoid precursor, is a desirable component in the formulation for treatment of rosacea at a concentration from 0.005% to 0.10%, preferably 0.05%.

In another embodiment, including a component in the formulation which inhibits inflammation is disclosed. Specifically, a non-antibiotic component which inhibits inflammation mediated by an interaction between cathelicidin peptides and stratum corneum tryptic enzyme (SCTE) may be used. Adding a non-antibiotic SCTE inhibitor to the topical formulation will increase overall efficacy and provide suppression of rosacea symptoms. SCTE inhibitors may be selected from aprotinin, chymostatin, zinc ions and combinations thereof. One or more of the formulation components for iron chelation may also be beneficial for SCTE inhibition. For example, zinc ions, either in the form of Zn²⁺ ions as chelated from the Zinc Omadine, or from the zinc salts, such as zinc sulfate and zinc acetate, added to prevent discoloration of the formulation, may already be present. Other SCTE inhibitors may be used as well.

The addition of humectants to the composition is optional but desirable. Such humectants aid in the rehydration and maintenance of hydration of the treated skin. In general, a humectant aids in increasing the effectiveness of an emollient; it may reduce scaling, stimulate removal of built-up scale, moisturize and improve skin feel. Examples of suitable humectants are: glycerin, propylene glycol, butylene glycol, diglycerol, or ester derivatives thereof, and sorbitol. The preferred humectants are sorbitol and glycerin.

Emollients are also optional but desirable in the invention. Examples of suitable emollients are: mineral oil, petrolatum, silicone, silicone-glycol copolymers, triglyceride esters, acetylated monoglycerides, alkyl esters of fatty acids, fatty acids and alcohols, lanolin and lanolin derivatives, beeswax derivatives, polyhydric alcohol, and amides of fatty acids. Although various emollients known in the art would be useful in the present invention, the preferred emollients are: mineral oil, petrolatum and silicone. Emollients may range from 0.10 to 10%, preferably from 0.5 to 7.0%.

The composition of the invention should be in a suitable vehicle. “A suitable vehicle” may be a cream, ointment, lotion or stick. Creams, lotions, ointments and sticks are well known in the prior art. It is preferred to formulate the ingredients in a vanishing cream base that is to be applied at least once a day.

While various emollients, humectants, antioxidants and sunscreen components, as well as other excipient ingredients including emulsifiers and stabilizers, may be employed in preparation of the formulation, it is important that only those components which are non-irritating to skin be selected. Patients suffering from rosacea have skin that is particularly prone to chemical insult and any composition to be used on areas with rosacea must have a low tendency to irritate or aggravate the condition. For example, sodium lauryl sulfate is a surfactant commonly used in shampoos and skin wash formulations, but which been found to be a skin irritant, particularly on prolonged contact and for those suffering from rosacea. Using a non-irritating surfactant, such as sodium laureth sulfate, is one embodiment of a formulation for treatment of rosacea. Irritation potential of ingredients can be tested individually prior to employment in a treatment formulation for rosacea. Such skin irritation tests are known in the art.

The composition of a preferred treatment formulation for use in a regimen for treating acne rosacea is exemplified below.

### Exemplary Formulation

### A. Lipid Phase

| INGREDIENT | AMOUNT |
|---|---|
| Lanolin | 50 grams |
| Vitamin E | 50 grams |
| Stearic Acid, Triple Pressed | 40 grams |
| Parsol 1789 (Avobenzone) | 30 grams |
| Zinc Oxide (Z-Cote) | 25 grams |
| White Petrolatum | 25 grams |
| Glycerol monolaurate | 20 grams |
| Emersol 305 or 315 | 20 grams |
| Retinaldehyde | 5 grams |
| Dimethicone | Optional as needed for function |
| Cyclomethicone | Optional as needed for function |

### B. Aqueous Phase

| INGREDIENT | AMOUNT | |
|---|---|---|
| Tenox S − 1* | 100 | cc |
| Sorbitol Solution (70%) | 50 | grams |
| Kojic acid dipalmitate | 5 | grams |
| Glycerin | 10 | grams |
| Zinc acetate | 10 | grams |
| Triethanolamine | 10 | grams |
| Deionized Water (USP) | Q.S. to 1000 | grams |

*80% Propylene Glycol, 20% Citric Acid and 10% Propyl Gallate.

Procedure: Heat the lipid phase and aqueous phase separately to 77-82° C., with constant stirring, until the contents of each part are solubilized. Add the lipid phase slowly to the aqueous phase while stirring until the contents of each part is solubilized. Continue stirring until the emulsion formed is uniform, and then cool the emulsion to 60° C. Add 41.67 cc of micro-fine Zinc Omadine (48%) suspension and blend rapidly. Fill jars while the emulsion is about 50° C., and then allow the jars of emulsion to cool to room temperature (22° C.).

An alternate formulation for use in a treatment regimen for acne rosacea employing both a Wash-off formulation and a Leave-on formulation is exemplified below.

### Exemplary Wash-Off Formulation

| | |
|---|---|
| Water | 88.57% |
| Propylene glycol | 3% |
| Sodium laureth sulfate | 2.5% |
| Cetyl alcohol | 2.5% |
| Zinc omadine | 2% |
| Stearyl alcohol | 0.5% |
| Acrylates/C10-30 alkyl acrylate crosspolymer | 0.1% |
| Phenoxyethanol | 0.392% |
| Sodium hydroxide | 0.25% |
| Sodium polynaphthalenesulfonate | 0.08% |
| Cellulose | 0.05% |
| Xanthan gum | 0.03% |
| Cellulose gum | 0.02% |
| Methylisothiazolinone | 0.008% |

### Exemplary Leave-On Formulation

| | |
|---|---|
| Water | 37.55% |
| Zinc oxide | 12% |
| Cyclomethicone | 7% |
| Glycerin | 6% |
| Kojic diplamitate | 5% |
| Isopropyl palmitate | 5% |
| Medium Chain Trigelycerides (MCT) oil | 3% |
| Polyglyceryl-2-diisostearate | 3% |
| Octyldodecyl neopentanoate | 3% |
| Sodium PCA | 3% |
| Prunus amygdalus dulcis (sweet almond oil) | 2.5% |
| Zinc laurate | 2% |
| Cetearyl alcohol | 2% |
| Ceteareth-20 | 2% |
| Cetyl alcohol | 2% |
| Oenothera biennis (evening primrose) oil | 1.75% |
| Glyceryl stearate | 1% |
| PEG-100 Stearate | 1% |
| Zinc omadine | 0.5% |
| Triethoxycaprylysilane | 0.3% |
| Sodium polynaphthalenesulfonate | 0.2% |
| Chondrus crispus (carrageenan) | 0.1% |
| Glucose | 0.1% |

### Example 1

A patient affected with rosacea is treated with a formulation comprising iron chelators which formulation is topically applied to the affected area. Preferably, the formulation is applied at least once daily to the affected area over a period of at least three weeks.

In an alternate embodiment a facewash formulation may be used in a regimen for treatment of rosacea. The facewash may comprise an iron chelator in a higher concentration than the leave-on formulation. The higher concentration is acceptable to skin since it will only be on the skin for a few minutes prior to being washed off.

### Example 2

Prior to retiring for the night, a patient with rosacea will apply to the affected area a rinse-off facewash formulation comprising Zinc Omadine at a concentration of 2.0%, leave the formulation in contact with the skin for one to five minutes, then gently but thoroughly rinse the formulation from the skin. The wait time is to allow for iron binding to occur. A greater efficacy may be seen when the formulation is left in contact with the affected area for five to fifteen minutes prior to rinsing. Upon rising in the morning, the patient will again apply the facewash formulation, then after one to five minutes gently but thoroughly rinse the formulation from the skin. Next, a sunscreen formulation containing from 0.25% to 0.5% Zinc Omadine in addition to emollient ingredients is applied to the skin. The patient will leave the topical formulation on the skin.

As an alternative, a leave-on formulation comprising one or more iron chelators, but which does not contain sunscreen, is applied to the affected area in a method for treating rosacea. Use of a formulation which does not contain sunscreen is preferred for individuals with sensitivities to such sunscreen components. This type of formulation may also be preferred when no sun exposure is anticipated, such as when the formulation is used to treat the skin at nighttime, for example, just prior to bedtime.

I claim:

1. A method for the treatment of acne rosacea comprising topically applying to a patient having a skin area in need of such treatment a formulation comprising an iron chelator and a topical carrier.

2. The method of claim 1, wherein said iron chelator is an alkaline earth metal salt of bispyrithone.

3. The method of claim 2, wherein the alkaline earth metal salt of bispyrithione is Zinc Omadine.

4. The method of claim 3, wherein the concentration of said Zinc Omadine in said formulation is from 0.0001% -5.0%.

5. The method of claim 4, wherein the concentration of said Zinc Omadine in said formulation is from 0.001% to 0.5%.

6. The method of claim 1, wherein said iron chelator is selected from kojic acid, kojic acid dipalmitate, kojic acid derivatives and combinations thereof.

7. The method of claim 6, wherein said concentration of said iron chelator is from about 0.5% -6.0%.

8. The method of claim 7, wherein the concentration of said iron chelator is from 1.0% -5.0%.

9. The method of claim 1, wherein said iron chelator is selected from the group consisting of Zinc Omadine, kojic acid, kojic acid dipalmitate, 1,10-phenanthroline, ethylenediaminetetraacetic acid (EDTA), 2-furildioxime, desferrioxamine, desferrithiocin, desferri-exochelin, deferiprone and hydroxypyridione analogs, tackpyridine, pyridoxyl isonicitinoyl hydrazone, triapine, 2-hydroxy-1-napthaldehyde-3-thiosemicarbazone and combinations thereof.

10. The method of claim 9, wherein said iron chelator is a combination of Zinc Omadine and kojic acid dipalmitate.

11. The method of claim 9, wherein concentration of said Zinc Omadine is from 0.001% to 5% and concentration of said kojic acid dipalmitate is from 0.5% to 6.0%.

12. The method of claim 1, wherein said formulation further comprises a medium-chain saturated fatty acid ester.

13. The method of claim 12, wherein the medium-chain saturated fatty acid ester is glycerol monolaurate.

14. The method of claim 13 wherein the concentration of glycerol monolaurate in said formulation is from 0.01% -5.0%.

15. The method of claim 14, wherein the concentration of glycerol monolaurate in said formulation is from 0.01% -2%.

16. The method of claim 1, wherein said formulation further comprises an unsaturated long-chain fatty acid selected from the group consisting of linoleic acid, gamma-dihomo-linolenic acid, oleic acid, elaidic acid and mixtures thereof.

17. The method of claim 16 wherein said unsaturated long-chain fatty acids are in hydrosylate form.

18. The method of claim 10, wherein the concentration of said unsaturated long-chain fatty acid in said formulation is from 0.01% -5.0%.

19. The method of claim 12, wherein the concentration of said unsaturated long-chain fatty acid in said formulation is from 0.1% -2.0%.

20. The method of claim 1, wherein said formulation further comprises a non-antibiotic inhibitor which inhibits inflammation.

21. The method of claim 20, wherein said inhibitor inhibits stratum corneum tryptic enzyme (SCTE).

22. The method of claim 21, wherein said inhibitor is selected from the group consisting of aprotinin, chymostatin, zinc ions and combinations thereof.

23. The method of claim 1, wherein said formulation further comprises one or more antioxidant agents.

24. The method of claim 23, wherein said antioxidant is selected from butylatedhydroxy toluene (BHT), alpha tocopherol, ascorbic acid and propyl gallate and combinations thereof.

25. The method of claim 24, wherein the concentration of said antioxidant in said formulation is an amount from 0.01% -5%.

26. The method of claim 1, further comprising a sunscreen agent.

27. The method of claim 1 where the formulation is a leave-on formulation which is applied to the affected areas of the skin from one to three times daily and left on the affected area without rinsing.

28. The method of claim 27 where the concentration of zinc pyrithione in said leave-on formulation is from 0.0001% to 0.5%.

29. The method of claim 27, further comprising a step precedent to said application of said leave-on formulation, said step precedent comprising application of a wash-off formulation to the affected area for 20 seconds to one minute followed by rinsing off said wash-off formulation.

30. The method of claim 29 where the concentration of zinc pyrithione in said wash-off formulation is from 1%-5%.

31. A topical formulation for treating a person afflicted with rosacea comprising one or more iron chelators, one or more medium-chain saturated fatty acid esters, an unsaturated long-chain fatty acid, and one or more antioxidant agents, in combination with effective sunscreen agents in a cosmetically acceptable carrier.

32. A topical formulation of claim 31, further comprising an inhibitor of stratum corneum tryptic enzyme (SCTE) selected from the group consisting of aprotinin, chymostatin, zinc ions and combinations thereof.
